(19) United States
(12) Patent Application Publication
(10) Pub. No.: US 2020/0337548 A1
(43) Pub. Date: Oct. 29, 2020

(54) LEADING-IN DEVICE OF FLEXIBLE URETEROSCOPE AND USING METHOD THEREOF AND URETERO-PYELOSCOPE SYSTEM

(71) Applicant: SIMAI CO., LTD., Zhuhai (CN)

(72) Inventors: Min Lin, Zhuhai (CN); Changsheng Yang, Zhuhai (CN); Mu Wang, Zhuhai (CN)

(21) Appl. No.: 16/860,075

(22) Filed: Apr. 28, 2020

(30) Foreign Application Priority Data
Apr. 29, 2019 (CN) 201910353948.8
Dec. 11, 2019 (CN) 201911263637.9
Dec. 11, 2019 (CN) 201911270216.9
Apr. 23, 2020 (CN) 202020641284.3

(51) Int. Cl. A61B 1/307; A61B 17/22; A61B 17/00

(52) U.S. Cl. CPC A61B 1/307; A61B 17/22; A61B 17/00234; A61B 2017/22078; A61B 2017/00238; A61B 2017/003; A61B 2017/0034

(57) ABSTRACT

The invention discloses a leading-in device of a flexible ureteroscope and a using method thereof and a uretero-pyeloscope system. The leading-in device of the flexible ureteroscope includes a leading-in sheath component, the leading-in sheath component includes a leading-in sheath with elasticity, and the leading-in sheath is a strip-type tube body matched with the flexible ureteroscope. While the flexible ureteroscope is inserted and connected in the leading-in sheath, an outer end part of the flexible ureteroscope is aligned with an outer end part of the leading-in sheath or slightly protruded out of the outer end part of the leading-in sheath. The invention is capable of enabling the flexible ureteroscope to be accurately inserted into a renal pelvis, putting in place by one step, using conveniently, reducing injury of a patient, and reducing the cost of an operation.

### CROSS REFERENCE TO RELATED APPLICATIONS

The present application claims the benefit of Chinese Patent Application Nos. 201910353948.8 filed on Apr. 29, 2019, 201911263637.9 and 201911270216.9 filed on Dec. 11, 2019, 202020641284.3 filed on Apr. 23, 2020. All the above are hereby incorporated by reference. the contents of which are incorporated herein by reference in their entirety.

### TECHNICAL FIELD

The invention relates to the technical field of medical apparatus and instruments, and in particular to a leading-in device of a disposable flexible ureteroscope and a using method thereof and a uretero-pyeloscope system.

### BACKGROUND

In recent years, a uretero-pyeloscope has become a commonly used auxiliary device for diagnosing and treating kidney diseases such as kidney stones, which changes a situation that a ureteral site is difficult to be visually inspected for a long time and a ureteral disease must be treated with an open-type operation. At present, there are separate flexible ureteroscope and inflexible ureteroscope on the market. The inflexible ureteroscope is convenient to operate, and may easily reach a renal pelvis through a ureter, However, because a front end of the inflexible ureteroscope is not flexible, it may not adjust a direction to enter various groups of renal calices for various diagnosis and treatment after entering the renal pelvis; and although a front end of the flexible ureteroscope may be mono-directionally or multi-directionally bended and turned at a large angle, it is difficult to enter the renal pelvis and the renal calices through the ureter. Although it may enter the renal pelvis and the renal calices under the guidance of an x-ray, the x-ray causes great damage to doctors and patients.

In an existing technology, the uretero-pyeloscope enters a body from a human urethra, and multiple repetitive operations are required to put the flexible ureteroscope in place: a first step, the inflexible ureteroscope with a lens is inserted to find a predetermined position from the human urethra, usually the predetermined position is a position where a lesion is located or a position close to the lesion, for example, reaching the ureter; a second step, a zebra guidewire is inserted into a position (may be observed through the lens) of the renal pelvis through a working channel of the inflexible ureteroscope; a third step, the inflexible ureteroscope is withdrew, but the zebra guidewire is kept in the position of the renal pelvis, until the inflexible ureteroscope is completely withdrew; a fourth step, a leading-in sheath component with a small round head is pushed to the position of the renal pelvis along the zebra guidewire (no lens monitoring at this moment, which belongs to blind insertion); a fifth step, a guiding sheath in the leading-in sheath component is pulled out, the zebra guidewire is pulled out too at the same time, and a leading-in sheath is kept in the human body; and a sixth step, the flexible ureteroscope is inserted to a portion of the renal pelvis along the leading-in sheath. This method greatly increases operating steps of a doctor and a degree of damage to an organ of a patent, the operation is complicated, the work efficiency is low, the operation cost is high, and the using effect is not good.

### SUMMARY

In order to overcome deficiencies of the existing technology, the invention aims to provide a leading-in device of a flexible ureteroscope. The leading-in device is capable of reducing operating steps of an operation, and improving work efficiency.

A technical scheme used by the invention is as follows.

A leading-in device of a flexible ureteroscope, the leading-in device includes a leading-in sheath component, the leading-in sheath component includes a leading-in sheath with elasticity, and the leading-in sheath is a strip-type tube body matched with the flexible ureteroscope. While the flexible ureteroscope is inserted and connected in the leading-in sheath, an outer end part of the flexible ureteroscope is aligned with an outer end part of the leading-in sheath or slightly protruded out of the outer end part of the leading-in sheath.

In the leading-in device, the flexible ureteroscope has functions of an inflexible ureteroscope, the leading-in sheath by which the flexible ureteroscope is carried enters a ureter to a renal pelvis orifice through a urethral canal, and then the leading-in sheath stops being pushed, the flexible ureteroscope is continuously pushed into a renal pelvis.

Preferably, an outer diameter of the leading-in sheath is 2-8 mm.

Preferably, the leading-in sheath component further includes a guiding sheath main body connected with the leading-in sheath, the flexible ureteroscope may pass through the guiding sheath main body and may be inserted in the leading-in sheath, an outer end part of the flexible ureteroscope is aligned with an outer end part of the leading-in sheath or slightly protruded out of the outer end part of the leading-in sheath, the leading-in sheath component further includes a fixing support for fixing the flexible ureteroscope, the fixing support is connected to the other end of the guiding sheath main body, and an inner end part of the flexible ureteroscope is detachably fixed on the fixing support.

Preferably, one end, adjacent to the leading-in sheath, of the guiding sheath main body is provided with a guiding sheath channel, the other end of the guiding sheath main body is provided with a groove through which the flexible ureteroscope passes, the groove is communicated with the guiding sheath channel, and the guiding sheath channel is communicated with the leading-in sheath.

Preferably, the guiding sheath main body is further provided with a water outlet channel, the water outlet channel is communicated with the guiding sheath channel.

Preferably, a position, close to the groove, on the guiding sheath channel is provided with a guiding sheath seal for sealing the flexible ureteroscope and the guiding sheath channel, and the guiding sheath seal is in interference fit with the guiding sheath channel.

Preferably, an end part of the guiding sheath main body is provided with a fixing rib, a corresponding end part of the fixing support is provided with a fixing button matched with the fixing rib, the fixing support is detachably connected with the guiding sheath main body through fastening of the fixing rib and the fixing button.

Preferably, a side face of the guiding sheath main body is provided with a guiding sheath handle.

The other purpose of the invention is to provide a using method for the above leading-in device.

The using method includes the following steps:

1) opening perfusion liquid;

2) a leading-in sheath of a leading-in sheath component by which the flexible ureteroscope is carried is inserted from a urethral orifice of a human body into a urethral canal; and

3) the leading-in sheath by which the flexible ureteroscope is carried successively enters a ureter and a renal pelvis orifice through the urethral canal, and then the leading-in sheath is stopped to be pushed, and the flexible ureteroscope is continuously pushed, until reaching a renal pelvis.

Another purpose of the invention is to provide a uretero-pyeloscope system.

The uretero-pyeloscope system includes a flexible ureteroscope and the above leading-in device, the flexible ureteroscope is inserted and connected in the leading-in sheath, an outer end part of the flexible ureteroscope is aligned with an outer end part of the leading-in sheath or slightly protruded out of the outer end part of the leading-in sheath.

In the uretero-pyeloscope system, the leading-in sheath is used for carrying the flexible ureteroscope and enters a ureter to a renal pelvis orifice through a urethral canal, and then the leading-in sheath is stopped to be pushed, and the flexible ureteroscope is continuously pushed until reaching a renal pelvis.

Preferably, the flexible ureteroscope includes a flexible ureteroscope main body and a flexible ureteroscope handle, the flexible ureteroscope main body includes a flexible tube and a lens component which is connected to an outer end part of the flexible tube through a bending part, an inner end part of the flexible tube is fixedly connected with the flexible ureteroscope handle, and the lens component is positioned on an outer end part of the flexible ureteroscope.

Preferably, an inner diameter size of the leading-in sheath is uniform, outer diameter sizes of the bending part and the lens component are greater than an outer diameter size of the flexible tube, the outer diameter size of the flexible tube is less than the inner diameter of the leading-in sheath and a radial size of the guiding sheath channel, and water passing clearances are formed between the flexible tube and the leading-in sheath, and between the flexible tube and the guiding sheath channel, liquid and operation fragments in the renal pelvis are discharged through the water passing clearances and the water outlet channel. Preferably, the outer diameter sizes of the bending part and the lens component are 2.5-4.5 mm, and the outer diameter size of the flexible tube is 1.5-4.0 mm.

Preferably, while the leading-in sheath component includes the fixing support, the flexible ureteroscope handle is detachably installed on the fixing support, the flexible ureteroscope main body is installed in the leading-in sheath, an outer end part of the lens component is aligned with an outer end part of the leading-in sheath or slightly protruded out of the outer end part of the leading-in sheath.

Preferably, the lens component includes a lens support, the flexible ureteroscope is internally provided with a working channel, the working channel successively passes through the flexible tube and the bending part from the flexible ureteroscope handle, and is stretched into the lens support, the lens support is provided with a through hole communicated with the working channel, the through hole is used for inputting the perfusion liquid; the lens component further includes an image pickup channel and an illumination channel which are installed in the lens support, the image pickup channel is internally provided with a camera device, the illumination channel is internally provided with an illumination device; the bending part includes a snake-like bone component and a fixing frame for fixing an end part of the snake-like bone component; and one end part of the snake-like bone component is connected with the flexible tube through the fixing frame, and another end part of the snake-like bone component is connected with the lens support.

Compared with the existing technology, the beneficial effects of the invention are as follows.

The invention provides a uretero-pyeloscope system, through enabling the flexible ureteroscope to be fixed on an inflexible leading-in sheath component, the flexible ureteroscope has the hardness and functions of the inflexible ureteroscope, so the flexible ureteroscope may reach the position of the renal pelvis rapidly; at the same time, the lens component, such as a camera, at the front end of the flexible ureteroscope is used for observing in real time, so that an inserting process is performed under endoscopy, and the accuracy and reliability of the operation may be further guaranteed; after the inflexible leading-in sheath component by which the flexible ureteroscope is carried enters the renal pelvis orifice, a character that the front end of the flexible ureteroscope may be controlled to be bended is used, the flexible ureteroscope enters the renal pelvis in complicated structure to examine and treat, in the whole process, the operating step of the doctor only needs one step, operative instruments are not required to be replaced, the operation is simple and rapid, the work efficiency is high, equipment used is less, disinfection steps are less, the loss is less, the operation cost is greatly reduced, and operation time is reduced, so the damage to the body of the patient is reduced and the probability of cross infection is reduced.

### BRIEF DESCRIPTION OF THE DRAWINGS

FIG. 1 is a structure diagram of a uretero-pyeloscope system;

FIG. 2 is a section view of the uretero-pyeloscope system;

FIG. 3 is a structure diagram of a flexible ureteroscope;

FIG. 4 is a structure diagram of connection of a lens support and a flexible ureteroscope main body;

FIG. 5 is an enlarged view of a part A in FIG. 4;

FIG. 6 is a structure diagram of the lens support;

FIG. 7 is a structure diagram of connection of a flexible tube and a flexible tube seal;

FIG. 8 is a structure diagram of connection of a bending part and the lens support;

FIG. 9 is a structure diagram of a water inlet connector;

FIG. 10 is a structure diagram of connection of a working channel and the water inlet connector;

FIG. 11 is an explosion view of a leading-in sheath component;

FIG. 12 is a section view of the leading-in sheath component; and

FIG. 13 is a structure diagram of a guiding sheath main body.

### DETAILED DESCRIPTION OF THE EMBODIMENTS

The invention is further described below in detail in combination with the drawings and the detailed description of the embodiments.

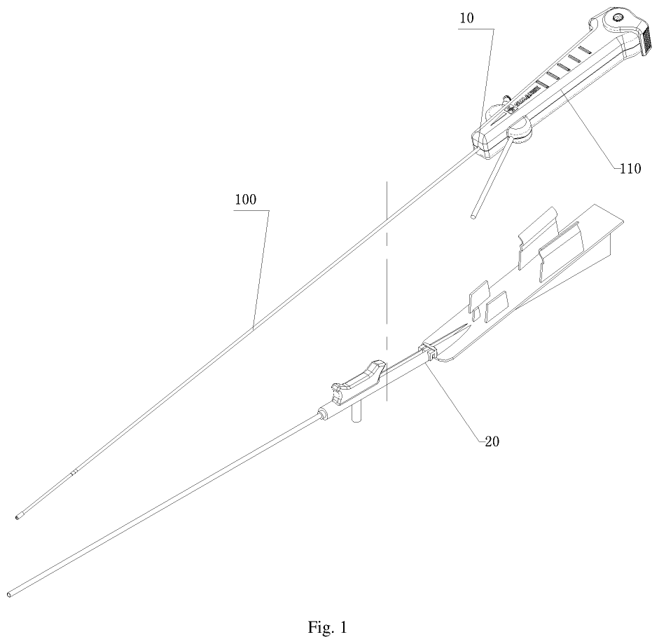

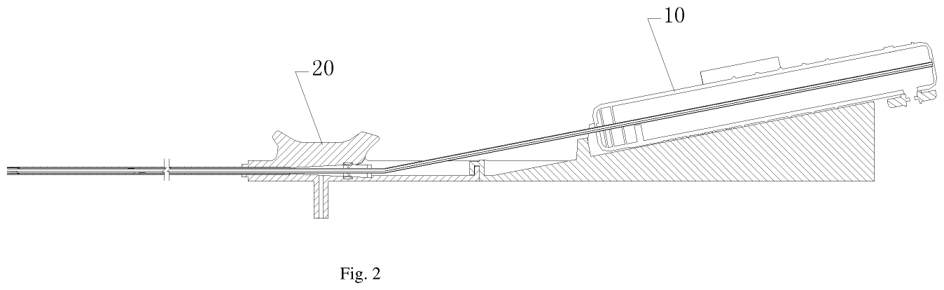

Reference to FIG. 1 and FIG. 2, the embodiment provides a disposable flexible uretero-pyeloscope system, the disposable flexible uretero-pyeloscope system includes a flexible ureteroscope 10 and a leading-in sheath component 20 served as a leading-in component, the flexible ureteroscope 10 is carried and fixed on the leading-in sheath component 20 and enters a renal pelvis tissue.

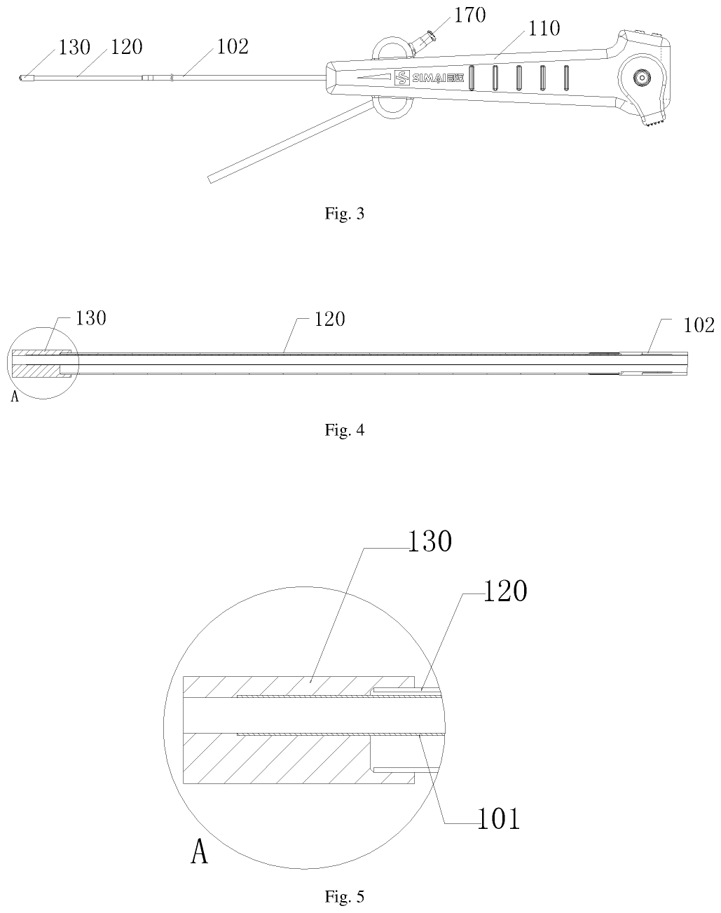

Specifically, as shown in FIG. 3, the flexible ureteroscope 10 includes a flexible ureteroscope main body 100 and a flexible ureteroscope handle 110 connected with the flexible ureteroscope main body 100, the flexible ureteroscope main body 100 includes a flexible tube 102, a bending part 120 and a lens support 130 which are connected successively.

As shown in FIG. 4 and FIG. 5, a structure of the flexible ureteroscope 10 is as follows: the flexible ureteroscope main body 100 is internally provided with a working channel 101; one end (namely one end of the flexible tube 102) of the flexible ureteroscope main body 100 passes through the flexible ureteroscope handle 110 and is fixedly connected with the flexible ureteroscope handle 110; the working channel 101 is inserted into the flexible tube 102 from a free end of the flexible ureteroscope handle 110, after passing through the flexible tube 102, stretched into the interior of the lens support 130 after passing through the bending part 120.

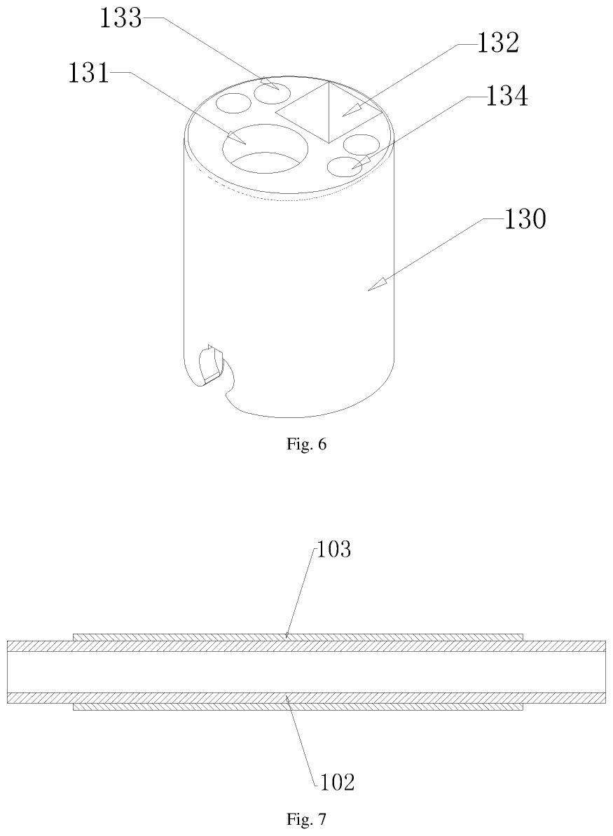

As shown in FIG. 6, the interior of the lens support 130 is provided with a through hole 131 communicated with the working channel 101, the through hole 131 is installed so as to input perfusion liquid. A position, corresponding to a lower part of the through hole 131, on the lens support 130 is provided with an image pickup channel 132, the image pickup channel 132 is internally provided with a camera device which is used for monitoring an operation process in real time. A position, corresponding to one side of the through hole 131, on the lens support 130 is provided with an illumination channel 133, the illumination channel 133 is internally provided with an illumination device, the illumination device is used for illuminating through a light transmitting bundle, the other opposite side of the illumination channel 133 is provided with two blind holes 134 which are used for installing a steering steel wire of the bending part 120. The lens support 130, the camera device and the illumination device form a lens component, and an outer diameter size of the lens component is the outer diameter size of the lens support 130.

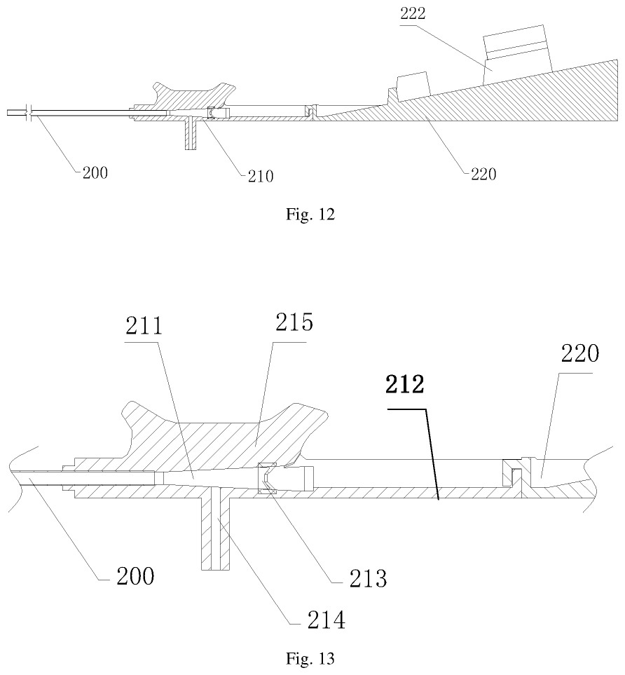

As shown in FIG. 11 and FIG. 12, the ureter leading-in sheath component 20 mainly includes a leading-in sheath 200, a guiding sheath main body 210 and a fixing support 220 for fixing the flexible ureteroscope 10 which are successively connected. The leading-in sheath 200 is a strip-type tube body, a structure thereof is matched with a structure of the straightened flexible ureteroscope main body 100, and the leading-in sheath 200 is made of metal or plastic, stronger in rigidity, has certain elasticity, and may be elastically deformed under the effect of an external force, one end of the leading-in sheath 200 is fixedly installed in the guiding sheath main body 210. While the flexible ureteroscope 10 is combined with the inflexible ureter leading-in sheath component 20 for using, the flexible ureteroscope main body 100 is inserted into the leading-in sheath 200 through the guiding sheath main body 210 and a head end of the flexible ureteroscope main body 100 is aligned or approximately aligned with a head end of the leading-in sheath 200, the flexible ureteroscope handle 110 is detachably installed on the fixing support 220. Herein the approximate alignment means that: the head end of the flexible ureteroscope main body 100 may be protruded out of the head end of the leading-in sheath 200 by 0-3 mm, it is the macroscopic alignment, and in order to avoid damage to inner walls of a urethral canal and a ureter, the head end of the leading-in sheath 200 is not protruded out of the head end of the flexible ureteroscope main body 100.

Specifically, as shown in FIG. 13, one end, close to the leading-in sheath 200, of the guiding sheath main body 210 is provided with a guiding sheath channel 211, the other end of the guiding sheath main body 210 is provided with a groove 212 through which the flexible ureteroscope main body 100 passes, the groove 212 is communicated with the guiding sheath channel 211. An end part of the leading-in sheath 200 is sealingly inserted and connected in the guiding sheath channel 211, an inner diameter of one end, away from the leading-in sheath 200, of the guiding sheath channel 211 is gradually increased towards a direction close to the groove 212. A position, close to the groove 212, on the guiding sheath channel 211 is provided with a guiding sheath seal 213, the guiding sheath seal 213 is used for sealing the flexible ureteroscope main body 100 and the guiding sheath channel 211, the guiding sheath seal 213 is in interference fit with the guiding sheath channel 211, and capable of effectively preventing the perfusion liquid from leaking outwards. A position, corresponding to an upper part of the guiding sheath channel 211, on the guiding sheath main body 210 is provided with a guiding sheath handle 215 which is conveniently grasped by a doctor, and an original using habit does not need to be changed. An end part, close to the groove 212, on the guiding sheath main body 210 is provided with a fixing rib 216, correspondingly, an end part of the fixing support 220 is provided with a fixing button 221 matched with the fixing rib 216, the fixing support 220 is detachably connected with the guiding sheath main body 210 through fastening of the fixing rib 216 and the fixing button 221. The fixing support 220 is provided with a flexible ureteroscope locking button 222, the flexible ureteroscope handle 110 is locked and connected on the flexible ureteroscope locking button 222, which has good fixing and positioning effects to the flexible ureteroscope 10. Reference to FIG. 1 and FIG. 2 at the same time, the flexible ureteroscope main body 100 is inserted into the leading-in sheath 200 through the groove 212 and the guiding sheath channel 211, a head end of the flexible ureteroscope main body 100 is aligned with a head end of the leading-in sheath 200, and the flexible ureteroscope handle 110 is locked and connected on the flexible ureteroscope locking button 222 of the fixing support 220, so that the flexible ureteroscope 10 is fixed on the inflexible leading-in sheath component 20.

The flexible tube 102 is preferably made by cutting a metal hard tube into an elastomer, that is a flexible metal tube is formed after an outer surface of the metal hard tube is spirally grooved along an axial direction thereof, the flexible tube 102 is guaranteed to have certain structural strength, and have good flexibility at the same time, so that the flexible ureteroscope main body 100 may achieve continuous bending. The flexible ureteroscope main body 100 further includes a flexible tube seal 103, the flexible tube seal 103 is installed on an outer surface of the flexible tube 102, and has sealing and protecting effects to the flexible tube 102. The flexible tube 102 and the flexible tube seal 103 may be sealed and fixed by using a mode of an adhesive, or a heat-shrink tube may be used as the flexible tube seal 103, the flexible tube seal 103 is fixed on the outer surface of the flexible tube 102 through a mode of heat shrinkage, it is sealed and fixed to effectively prevent the flexible tube 102 from being corroded.

The bending part 120 has at least one degree of freedom of oscillation, so a bending direction thereof may be adjusted by a steering steel wire, so that an entrance of the working channel 101 accurately reach a position of a renal pelvis, this requires that the bending part 120 may be bended randomly. In the embodiment, the bending part 120 includes a snake-like bone component and a fixing frame 121 for fixing an end part of the snake-like bone component, the specific connection relationship is as follows:

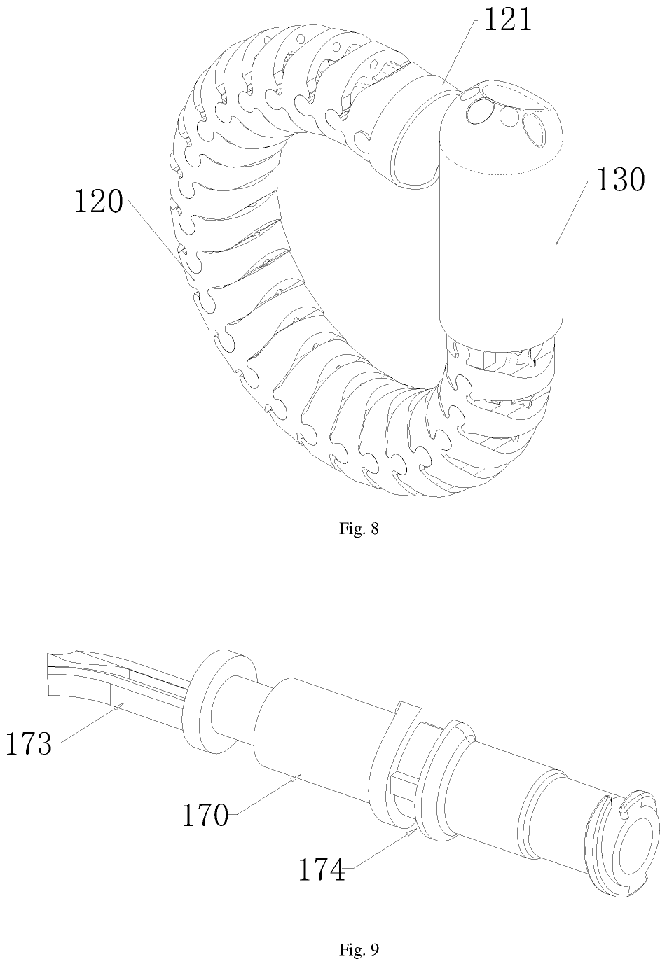

As shown in FIG. 8, one end part of the snake-like bone component is connected with the flexible tube 102 through the fixing frame 121, and the another end part of the snake-like bone component is connected with the lens support 130, thereby a steering function of the lens support 130 and the working channel 101 is realized.

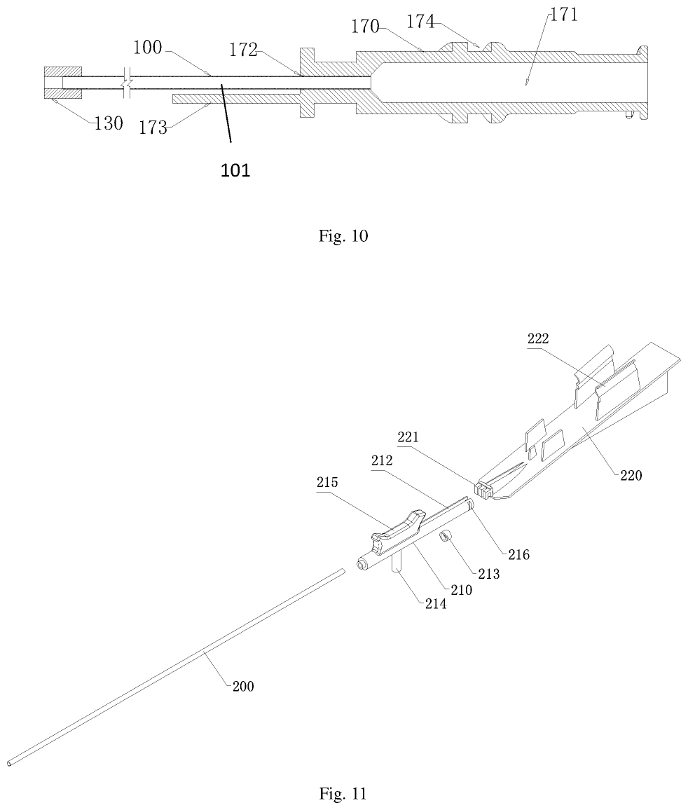

As shown in FIGS. 3, 9 and 10, in the embodiment, the working channel 101 is fixedly installed in the flexible ureteroscope handle 110 through a water inlet connector 170. The water inlet connector 170 includes a connector main body with a connecting cavity 171, the connector main body is internally provided with a connecting channel 172 for connecting the working channel 101, and the connecting channel 172 is communicated with the connecting cavity 17. One end, close to the connecting channel 172, of the connector main body is provided with a bearing plate 173, the bearing plate 173 is provided with a groove which is used for bearing the working channel 101. An outer circumferential surface of the connector main body is provided with at least one locking slot 174, the water inlet connector 170 is locked and connected on the flexible ureteroscope handle 110 through the locking slot 174.

One preferable implementation mode is as shown in FIG. 13, the guiding sheath main body 21 is further provided with a water outlet channel 214, the water outlet channel 214 is communicated with the guiding sheath channel 211, an inner diameter size of the leading-in sheath 200 is uniform, the outer diameter sizes of the bending part 120 and the lens component are both greater than the outer diameter size of the flexible tube 102, so the outer diameter sizes of the bending part 120 and the lens component are matched with the inner diameter size of the leading-in sheath 200, and the outer diameter size of the flexible tube 102 is less than the inner diameter size of the leading-in sheath 200 and a radial size of the guiding sheath channel 211, and water passing clearances are formed between the flexible tube 102 and the leading-in sheath 200, and between the flexible tube 102 and an inner wall of the guiding sheath channel 211, liquid and operation fragments in the renal pelvis are discharged through the water passing clearances and the water outlet channel 214. Specifically, the outer diameter sizes of the bending part 120 and the lens component 130 are 2.5-4.5 mm, and the outer diameter size of the flexible tube 102 is 1.5-4.0 mm, lengths of the bending part 120 and the lens component are both within a range of 35-100 mm, and a length of the flexible tube 102 is between 550 mm and 650 mm. A purpose of this design is as follows: on one aspect, the outer diameter sizes of the bending part 120 and the lens component are matched with the inner diameter size of the leading-in sheath 200, so that a radial clearance between a head end of the flexible ureteroscope and a head end of the leading-in sheath is smaller, an apparent step may not be formed, pain of the patient is avoided from being increased during insertion from a human urethral canal, and in a process of the insertion from the human urethral canal, relative stability between the head end of the flexible ureteroscope and the head end of the leading-in sheath may be guaranteed; on another aspect, while the leading-in sheath component is stopped to be pushed, the flexible ureteroscope is continuously pushed, until the lens component and the bending part 120 are stretched into the renal pelvis for performing a lithotripsy, as a radial size of the flexible tube 102 is smaller, so clearances between the flexible tube 102 and the leading-in sheath 200 and the guiding sheath channel 211 are larger, it is beneficial to discharge the liquid injected into the renal pelvis and crushed calculi through the water outlet channel 214, a convection current is formed with a water inlet channel and a water flow rate may be adjusted, it not only has the effects of convection washing to an object in a cavity and crushed calculi discharging, but also prevents a pressure in the cavity from being too large, so that the liquid is backwards flowed to cause urosepsis, and even endanger the life of the patient. If a rubber layer is sleeved on the bending part 120, the outer diameter size of the rubber layer is greater than the outer diameter size of the flexible tube 102.

After the flexible ureteroscope 10 and the leading-in sheath component 20 are fixed, a using method for the leading-in device specifically includes the following steps:

1) opening the perfusion liquid, opening the camera device and the illumination device, so a display screen connected with the flexible ureteroscope 10 displays a real-time image at the front end of the lens support 130 in real time;

2) the leading-in sheath 200 of the leading-in sheath component 20 is inserted from a urethral orifice of a human body into a urethral canal, and the flexible ureteroscope main body 100 is placed in the body along with the leading-in sheath 200 at the same time: the doctor only needs to grasp the guiding sheath handle 215 and enable the leading-in sheath 200 and the flexible ureteroscope main body 100 to be inserted into the body of the patient, in an inserting process, a situation of the whole inserting process is observed through the display screen, and direction and speed of inserting are adjusted in time; and

3) after passing through a bladder, finding out an entrance of a lower end of the ureter, and continuously pushing in the ureter by a certain distance until reaching the renal pelvis orifice, the leading-in sheath component 20 is stopped to be forwards pushed and pre-fixed, the flexible ureteroscope handle 110 on the fixing support 220 is taken down, the flexible ureteroscope main body 100 is continuously forwards pushed along the leading-in sheath 200, and enters the renal pelvis, so the examination or operation may be performed.

Thus It may be seen from this, the invention is capable of, through enabling the flexible ureteroscope 10 to be fixed on the leading-in sheath component 20, enabling the flexible ureteroscope 10 to be carried on the leading-in sheath component 20, compared with an existing pyeloscope, it only needs one-step operation to accurately reach the position of the renal pelvis, the operation is simple and rapid, the work efficiency is greatly improved, equipment, such as a zebra guiding wire, is not required, the equipment used is less, the operation cost is effectively reduced, operation time is saved, and the damage to the patient is reduced. At the same time, because the camera device and the illumination device are always installed at the front end of the lens support 130, the whole inserting process and operation process are visible.

The principle and structure of the soft-hard combined uretero-pyeloscope system used in the invention may also be applied to other endoscopes, such as a cystoscope, an enteroscope or a hysteroscope, and an application range is wider.

The above implementation modes are only preferable implementation modes of the invention, and may not be used to limit a scope of protection of the invention, any non-substantial changes and replacements made by those skilled in the art on the basis of the invention shall fall within the scope of protection required by the invention.

What is claimed is:

1. A leading-in device of a flexible ureteroscope, wherein the leading-in device comprises a leading-in sheath component (20), the leading-in sheath component (20) comprises a leading-in sheath (200) with elasticity, and the leading-in sheath (200) is a strip-type tube body matched with the flexible ureteroscope, while the flexible ureteroscope is inserted and connected in the leading-in sheath (200), an outer end part of the flexible ureteroscope is aligned with an outer end part of the leading-in sheath (200) or slightly protruded out of the outer end part of the leading-in sheath (200).

2. The leading-in device of the flexible ureteroscope as claimed in claim 1, wherein the leading-in sheath component (20) further comprises a guiding sheath main body (210) connected with the leading-in sheath (200), the flexible ureteroscope may pass through the guiding sheath main body (210) and may be inserted in the leading-in sheath (200), the leading-in sheath component (20) further comprises a fixing support (220) for fixing the flexible ureteroscope, the fixing support (220) is connected to one end, away from the leading-in sheath (200), of the guiding sheath main body (210), and an inner end part of the flexible ureteroscope is detachably fixed on the fixing support (220).

3. The leading-in device of the flexible ureteroscope as claimed in claim 2, wherein one end, adjacent to the leading-in sheath (200), of the guiding sheath main body (210) is provided with a guiding sheath channel (211), another end of the guiding sheath main body (210) is provided with a groove (212) through which the flexible ureteroscope passes, the groove (212) is communicated with the guiding sheath channel (211), and the guiding sheath channel (211) is communicated with the leading-in sheath (200).

4. The leading-in device of the flexible ureteroscope as claimed in claim 3, wherein the guiding sheath main body (210) is further provided with a water outlet channel (214), the water outlet channel (214) is communicated with the guiding sheath channel (211).

5. A uretero-pyeloscope system, wherein the uretero-pyeloscope system comprises a flexible ureteroscope (10) and the leading-in device as claimed in claim 1, the flexible ureteroscope (10) is inserted and connected in the leading-in sheath (200), an outer end part of the flexible ureteroscope (10) is aligned with an outer end part of the leading-in sheath (200) or slightly protruded out of the outer end part of the leading-in sheath (200).

6. The uretero-pyeloscope system as claimed in claim 5, wherein the flexible ureteroscope (10) comprises a flexible ureteroscope main body (100) and a flexible ureteroscope handle (110), an inner end part of the flexible ureteroscope main body (100) is fixedly connected with the flexible ureteroscope handle (110), the flexible ureteroscope main body (100) comprises a flexible tube (102) and a lens component which is connected to an outer end part of the flexible tube (102) through a bending part (120).

7. The uretero-pyeloscope system as claimed in claim 6, wherein outer diameter sizes of the bending part (120) and the lens component are greater than an outer diameter size of the flexible tube (102), the outer diameter size of the flexible tube (102) is less than an inner diameter of the leading-in sheath (200) and a radial size of the guiding sheath channel (211), and water passing clearances are formed between the flexible tube (102) and the leading-in sheath (200), and between the flexible tube (102) and the guiding sheath channel (211).

8. The uretero-pyeloscope system as claimed in claim 6, wherein while the leading-in sheath component (20) comprises the fixing support (220), the flexible ureteroscope handle (110) is detachably installed on the fixing support (220), the flexible ureteroscope main body (100) is installed in the leading-in sheath (200), an outer end part of the lens component is aligned with an outer end part of the leading-in sheath (200) or slightly protruded out of the outer end part of the leading-in sheath (200).

9. The uretero-pyeloscope system as claimed in claim 8, wherein the lens component comprises a lens support (130), the flexible ureteroscope (10) is internally provided with a working channel (101), the working channel (101) successively passes through the flexible tube (102) and the bending part (120) from the flexible ureteroscope handle (110) into the lens support (130), the lens support (130) is provided with a through hole (131) communicated with the working channel (101), the lens component further comprises an image pickup channel (132) and an illumination channel (133) which are installed in the lens support (130), the image pickup channel (132) is internally provided with a camera device, the illumination channel (133) is internally provided with an illumination device; the bending part (120) comprises a snake-like bone component and a fixing frame (121) for fixing an end part of the snake-like bone component; and one end part of the snake-like bone component is connected with the flexible tube (102) through the fixing frame (121), and the another end part of the snake-like bone component is connected with the lens support (130).

10. A using method for the leading-in device as claimed in claim 1, wherein the method comprises the following steps:
1) opening perfusion liquid;
2) enabling a leading-in sheath (200) of a leading-in sheath component (20) by which the flexible ureteroscope (10) is carried to be inserted from a urethral orifice of a human body into a urethral canal; and
3) enabling the leading-in sheath (200) by which the flexible ureteroscope (10) is carried to successively enter a ureter and a renal pelvis orifice through the urethral canal, and then stopping pushing the leading-in sheath (200), and continuously pushing the flexible ureteroscope (10), until it stretched into a renal pelvis.
